(19) United States
(12) Patent Application Publication
(10) Pub. No.: US 2023/0190362 A1
(43) Pub. Date: Jun. 22, 2023

(54) APPARATUS FOR THE CONNECTION OF ELECTROSURGICAL INSTRUMENT ASSEMBLIES

(71) Applicant: GYRUS MEDICAL LIMITED, Cardiff (GB)

(72) Inventors: Teo Heng Jimmy YANG, Cardiff (GB); Marno NAGTEGAAL, Cardiff (GB)

(73) Assignee: GYRUS MEDICAL LIMITED, Cardiff (GB)

(21) Appl. No.: 18/083,063

(22) Filed: Dec. 16, 2022

Related U.S. Application Data
(60) Provisional application No. 63/291,792, filed on Dec. 20, 2021.

(51) Int. Cl. A61B 18/14

(52) U.S. Cl. CPC A61B 18/148; A61B 2018/1226

(57) ABSTRACT

A surgical instrument is configured to consist of two separable parts. The first part includes a disposable end effector unit, including an electrosurgical end effector and mechanical drive componentry. The second part includes a reusable electronics unit, containing the RF electronic circuitry of the instrument. A user-releasable complementary interface is provided on each part, allowing the parts to be connected in use. Providing a division of components in this manner improves the sterility of the device, providing easier cleaning of the reusable part of the instrument.

### TECHNICAL FIELD

Embodiments of the present invention described herein relate to an electrosurgical instrument, including two parts. In particular, embodiments of the present invention define the connection and interface between the two parts of the instrument so as to improve sterility of the instrument between surgical operations.

### BACKGROUND TO THE INVENTION

During use, a surgical instrument may be used to cut, ablate, or alter tissue inside of a patient. This may cause free-moving blood, tissue or other surgical debris from the patient to attach itself to the exterior, or some interior sections, of the instrument, contaminating the instrument. Due to the design of some surgical instruments, ensuring that the device is sterile for subsequent use may be difficult, due to challenges in cleaning the parts. Some electrosurgical instruments are constructed from a number of parts, some of which may be reusable and some of which may be single use. Following surgery, disposable sections of the instrument may be discarded, and any reusable parts may be cleaned so as to remove, for example, surgical debris. The design of the surgical instrument may present a number of challenges in ensuring that the instrument is sterile following use. If a device is not correctly sterilised, there may be poorer patient outcomes due to cross-contamination risk.

For example, one such prior art arrangement is disclosed in EP 2 098 170 B1, which describes a surgical instrument with a number of interchangeable surgical tool attachments that may be used with a surgical instrument hand piece. The surgical tool attachments are connected to the hand piece using a control rod, allowing a clinician to operate the surgical tool attachments with a movable handle which forms part of the surgical instrument hand-piece. In some instances, where the surgical tool attachment is designed to cauterize tissue, the surgical tool attachment may include a battery. The moveable handle of the surgical hand-piece may drive battery contacts of the surgical tool attachment closed, providing electrical power to the cauterizing section.

Another prior art arrangement is disclosed in U.S. Pat. No. 10,039,529 B2, which describes a surgical instrument with an end-effector controlled by a pivotable trigger attached to a handle. The surgical instrument includes a battery dock, which allows a battery unit to be inserted into the surgical instrument.

A final prior art arrangement is disclosed in EP 3 222 223 B1, which describes a cordless motor-powered surgical instrument, which may be powered by a power pack containing a DC power source, such as one or more series-connected battery cells. The power pack may be removable from the instrument and connectable to a remote charger base.

So as to improve upon the prior art, the present disclosure seeks to provide an improved surgical instrument which provides a simpler, more sterile system for use in surgery.

### SUMMARY OF INVENTION

Embodiments of the present invention provide an improved surgical instrument consisting of two assemblies. A first assembly includes an end effector, a mechanical drive for the end effector and an interface. A second assembly includes electronic circuitry and an interface. The interfaces of the first and second assemblies may be arranged such that the first and second assemblies can be connected along the complementary interfaces to form the electrosurgical instrument. So as to improve sterilisation and reusability of the electrosurgical instrument, the components of the electrosurgical instrument are arranged between the two assemblies in a manner that reduces the number of connections required between the two assemblies along the interfaces. This simplifies the sterilisation procedure required, especially for the second assembly, which may have few or no apertures, reducing the potential for blood or tissue to enter the second assembly during a surgical procedure. Not only does this reduce the risk of cross-contamination between patients, but it ensures that complex electronic circuitry is reused and is not considered to be a single-use component. This is both more environmentally friendly, reducing waste, whilst also being safe for use in a surgical environment.

The first assembly may include all moving parts of the electrosurgical instrument, whilst the second assembly may include radio frequency generator circuitry and a battery. This may result in no transfer of mechanical motion between the two assemblies, allowing the second assembly, the electronics unit, to be a fully sealed device, having only external electrical contacts. Sealing the second assembly prevents biological matter or surgical debris from entering the second assembly, resulting in only surface sterilisation being required for the second assembly.

In view of the above, in a first aspect of the present disclosure there is provided a reusable electronics unit for use in an electrosurgical instrument, the electrosurgical instrument in use comprising an end effector unit including an end effector, and the reusable electronics unit, the reusable electronics unit comprising: a radio frequency (RF) electrosurgical signal generator circuitry for supplying in use RF electrosurgical signals to the end effector; wherein the reusable electronics unit is sealed, the reusable electronics unit further comprising a user-releasable electrical interface connectable in use to a complementary electrical interface provided on the end effector unit through which the RF electrosurgical signals are supplied in use.

The reusable electronics unit may be hermetically sealed.

The reusable electronics unit may further comprise a battery.

The reusable electronics unit may further comprise inductive charging circuitry, such that the battery of the reusable electronics unit may be charged inductively.

There may be no mechanical drive between the reusable electronics unit and the end effector unit.

In a second aspect of the present disclosure there is provided an end effector unit for use in an electrosurgical instrument, the electrosurgical instrument in use comprising the end effector unit and a sealed reusable electronics unit, the reusable electronics unit including a radio frequency (RF) electrosurgical signal generator circuitry for supplying in use RF electrosurgical signals to the end effector, the end effector unit comprising: an end effector; wherein the end effector unit further comprises a user-releasable electrical interface connectable in use to a complementary electrical interface provided on the reusable electronics unit through which the RF electrosurgical signals are supplied in use.

The end effector unit may further comprise a movable mechanism capable of receiving user input, such that the operation of the electrosurgical end effector is altered by movement of the mechanism.

The end effector unit may be a single-use unit.

There may be no mechanical drive between the reusable electronics unit and the end effector unit.

In a third aspect of the present disclosure there is provided an electrosurgical instrument, comprising: the reusable electronics unit according the above; and the end effector unit according to the above, wherein the interface of the reusable electronics unit is connected to the interface of the end effector unit.

All moving parts of the electrosurgical instrument may be contained within the unitary disposable end effector unit.

In a fourth aspect of the present disclosure there is provided an electrosurgical instrument comprising a first part and a second part, the first part being permitted a limited number of uses and the second part being re-usable, the first part comprising an end effector including one or more electrosurgical electrodes, and associated mechanical actuation componentry including user-operable componentry to control the end effector via the associated mechanical actuation componentry, the arrangement being such that the first part is not sealed such that it is capable of being contaminated internally by surgical debris when the end effector is in use; the second part comprising RF electrosurgical signal generation circuitry for supplying in use RF electrosurgical signals to the electrosurgical electrodes of the end effector, the second part being sealed to prevent the ingress of surgical debris when in use to prevent internal contamination by the surgical debris.

The first part may be single-use.

There may be no mechanical drive between the first part and the second part.

The second part may be hermetically sealed.

### BRIEF DESCRIPTION OF THE DRAWINGS

Embodiments of the invention will now be further described by way of an example only and with reference to the accompanying drawings, wherein like reference numerals refer to like parts, and wherein:

FIG. 1 is a diagram of an electrosurgical instrument, outlining the reusable and disposable sections of the instrument.

FIG. 2 is a diagram of an electrosurgical instrument, separated into two individual parts.

FIG. 3 is a diagram of an electrosurgical instrument, with the two individual parts in the process of being connected.

FIG. 4 is a diagram of an electrosurgical instrument, with the two individual parts fully connected.

### DESCRIPTION OF THE EMBODIMENTS

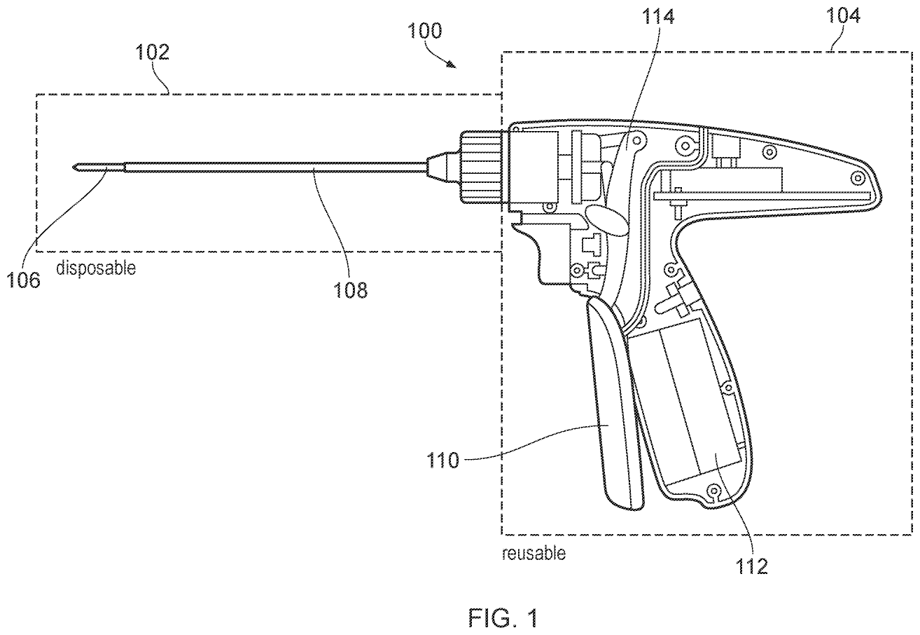

Referring to the drawings, FIG. 1 shows a traditional electrosurgical instrument 100, which is formed of multiple components. Section 102 of the electrosurgical instrument 100 is disposable following a single surgical procedure or a limited number of surgical procedures, whilst section 104 of the electrosurgical instrument may be reusable between surgical procedures. So as to prevent cross-contamination, the reusable section 104 of the electrosurgical instrument must be sterilised between uses of the instrument.

In some designs, the disposable part 102 of the electrosurgical instrument 100 includes the electrosurgical end effector only. For example, where the instrument is a vessel sealing device, the disposable part may include a jaw assembly, 106, used for clamping and sealing tissue and a shaft 108. The reusable section 104 may include a movable handle 110, a mechanical linkage 114, which transfers the movement of the handle such that the operation or position of the end effector is altered by the movement and, where the device is a cordless or wireless device, a battery 112.

However, a division of components between the reusable 102 and single use 104 sections of the electrosurgical instrument such as this may present difficulties in ensuring proper sterilisation of the device between uses. For example, during surgery the electrosurgical instrument may be contaminated by blood and tissue debris from a patient. A new single-use disposable section 102 of the instrument 100 may be used for each surgical procedure, therefore there is no issue with sterility of the disposable section. However, blood or tissue debris may transition into the reusable section 104 of the instrument during use, for example through apertures at the boundary between the two sections. It may be difficult to sterilise movable parts of the reusable section 104 of the surgical instrument 100. For example, sterilisation may require a clinician to dismantle the reusable section of the surgical instrument.

Improving the arrangement of the surgical instrument 100 may therefore provide an improved surgical instrument.

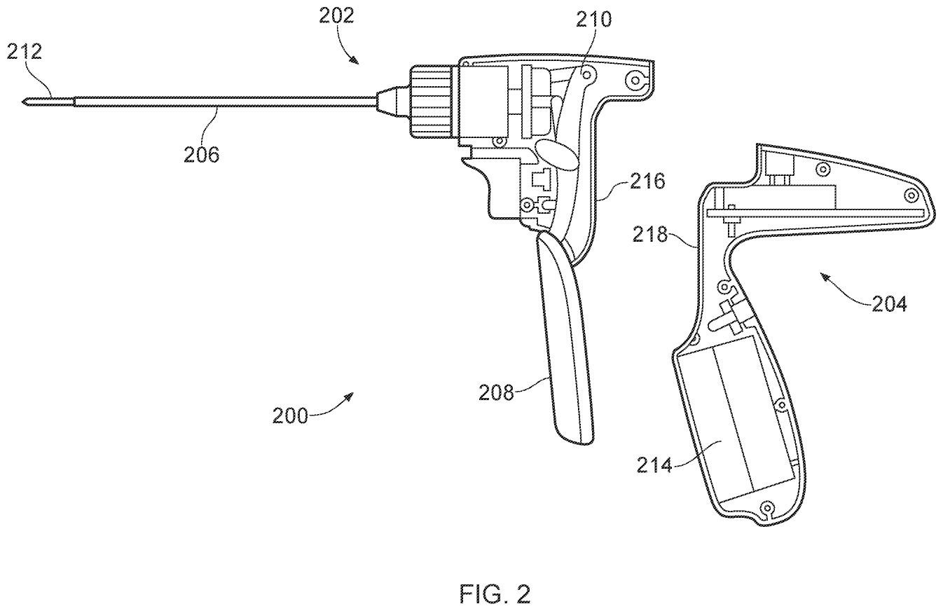

FIG. 2 shows a disassembled electrosurgical instrument 200. The instrument 200 comprises two parts, a first part 202 and a second part 204. The first part may be an end effector unit 202 and the second part may be an electronics unit 204. The end effector unit 202 may be disposable or single-use, for example it may be used for only a single surgical procedure before it is disposed of. Alternatively, it may be used for a limited number of surgical procedures before being disposed of. The electronics unit 204 may be reusable, for example it may be used for a number of surgical procedures, on a number of patients.

The electronics unit 204 of the electrosurgical instrument contains radio frequency, RF, electrosurgical signal generator circuitry, arranged such that during use it may provide an RF electrosurgical signal or RF electrosurgical energy. Radio frequency signals such as these are used in surgery, for example to cut or ablate tissue. The electrosurgical signal may be supplied to one or more electrodes located on the end effector.

The end effector unit 202 of the electrosurgical instrument 200 comprises an end effector 212. During use, the electrosurgical end effector 212 may apply an RF signal to the patient's tissue, cutting or ablating the tissue. The end effector may further comprise mechanical actuation or drive componentry 210 for the end effector, arranged such that movement of the mechanical drive componentry 210 alters the operation of the end effector. However, in some instances the end effector may not comprise mechanical drive componentry, and instead may include only the end effector. For example, the end effector may be a fixed monopolar or bipolar electrode arrangement, which may be referred to as an elongation instrument. The end effector unit 202 may further comprise a movable mechanism 208, arranged so as to receive user input. The movable mechanism 208 may be a trigger or handle mechanism, arranged such that when the movable mechanism 208 is moved, the operation of the electrosurgical end effector is altered. The movable mechanism may be any form of mechanism capable of receiving user input and being user-operable. The movable mechanism may be connected to the mechanical drive componentry 210, which may be a mechanical linkage 210, which extends down the shaft 206 of the electrosurgical end effector and is connected to the distal end of the end effector. Where the end effector 212 is, for example, a jaw assembly for clamping and cutting or sealing tissue, the movement of the movable mechanism 208 may cause the jaws of the jaw assembly to open or close.

Where the electrosurgical instrument 200 is a cordless or wireless device, the electronics unit 204 may include a battery. The battery 214 may provide power to the RF electronics, such that no external power connection is required. The usability of the device may be improved by virtue of its cordless nature, with no cables in the way to hinder a clinician's movements.

Dividing the component parts of the electrosurgical instrument 200 in the way defined in FIG. 2 improves sterility of the device. The disposable section 202 of the electrosurgical instrument may contain the moving parts of the instrument, for example, the movable mechanism 208 and the electrosurgical end effector 212. During a surgical procedure, blood or tissue may migrate inside the end effector unit 202, which may be contaminated internally by surgical debris when in use, due to, for example, a large number of moving mechanical linkages 210. Seals arranged around these linkages may be imperfect, or not practical to include in some areas. If the end effector unit 202 were sterilised as a single assembly following use, blood or tissue matter may remain in hard-to-reach areas. This may pose a cross-contamination risk if used with a different patient. If the end effector unit 202 was taken apart to sterilise each constituent component that makes up the end-effector unit 202, the clinician or surgeon may be required to perform a complex deconstruction, sterilisation, and reconstruction procedure. This may substantially increase the time take to sterilise the device, reducing efficiency and still providing potentially poor sterilisation outcomes.

Instead, the end effector unit 202 is a limited-use or single-use disposable unit, removing or reducing the sterilisation requirements that may exist otherwise.

So as to prevent the disposal of the entire electrosurgical instrument 200 after each use, the electronics unit may be a reusable unit. The electronics unit comprises the RF electronic circuitry. This is less likely to be contaminated by blood or tissue during a surgical procedure, as electronic RF circuitry comprises no moving parts.

Notably, dividing the components between the reusable 204 and single-use 202 sections of the electrosurgical instrument 202 in the manner described simplifies the interface between the two sections. The end effector unit 202 includes an interface 216 and the electronics unit includes an interface 218.

For example, the electronics unit 204 includes no moving parts, or at least includes no parts in which motion must be transferred to the end effector unit 202 during operation of the instrument across the interfaces 216, 218. There is no mechanical drive between the electronics unit and the end effector unit. This allows a reduced number of connections between the electronics unit and end effector unit of the electrosurgical instrument 200 when compared to prior art surgical instruments. Reducing apertures or connections along the interfaces 216, 218, between the two sections reduces the number of potential ingress points, which may allow blood or tissue matter to enter the reusable section 204 of the apparatus. As there is a lower likelihood of ingress, the sterilisation of the electronics unit 204 is simple when compared to a unit which includes moving parts.

As there are no moving parts in the electronics unit 204, only electrical signals, for example RF electrosurgical signals, are passed between the electronics unit 204 and the end effector unit 202. This may be achieved, for example, using a pair of electrical contacts located on the interface 218 of the electronics unit 204, which connect, or come into contact with, electrical contacts located on the interface 216 of the end effector unit 202. These electrical contacts do not require apertures or mechanical drives to be located between the two units, for example along the interfaces 216, 218, reducing the likelihood of blood or tissue entering the electronics unit 204.

Including both the battery 214 and the RF circuit in the electronics unit 204 of the electrosurgical instrument 200 further reduces the number of connections required between the end effector unit 202 and the electronics unit 204, such that only a pair of electrical contacts may be required. This single assembly reduces the number of exposed wires or electrical contacts required between the two units, reducing the likelihood of electrical shorts when the instrument comes into contact with saline or bodily fluids.

Further, as the surgical instrument is made up of only two units, the electronics unit 204 and the end effector unit 202, assembly of the instrument 200 is simple for a clinician, compared to a device made up of a large number of sections.

Inductive charging circuitry may be included in the electronics unit 204, allowing the battery 214 of the electronics unit to be charged or recharged wirelessly. This allows the electronics unit 204 to be reused a large number of times, and the life of the electronics unit 204 is not limited to the life of a single-use battery. Further, using a wireless charging mechanism further reduces the number of apertures or electrical contacts required on the surface of the electronics unit. This improves sterilisation and reduces the chances of short-circuits due to, for example, saline or bodily fluid.

The electronics unit 204 may be a sealed unit, for example a partially sealed unit or a hermetically sealed unit, with no external apertures. This prevents the ingress of any blood, tissue, or other surgical debris when in use, preventing internal contamination of the unit. Only external sterilisation of the electronics unit 204 may required, both improving and simplifying the sterilisation procedure. The electronics unit 204 may therefore be sterilised using cold plasma or an autoclave. In contrast, the end effector unit may be susceptible to internal contamination, due to its moving parts or mechanical actuation componentry. It may not be possible to seal the end effector unit, as it may have a number of external moving mechanical inputs or outputs, for example the end effector may move to cut tissue and there may be means for user input. This increases the likelihood of internal contamination of the end effector unit by surgical debris.

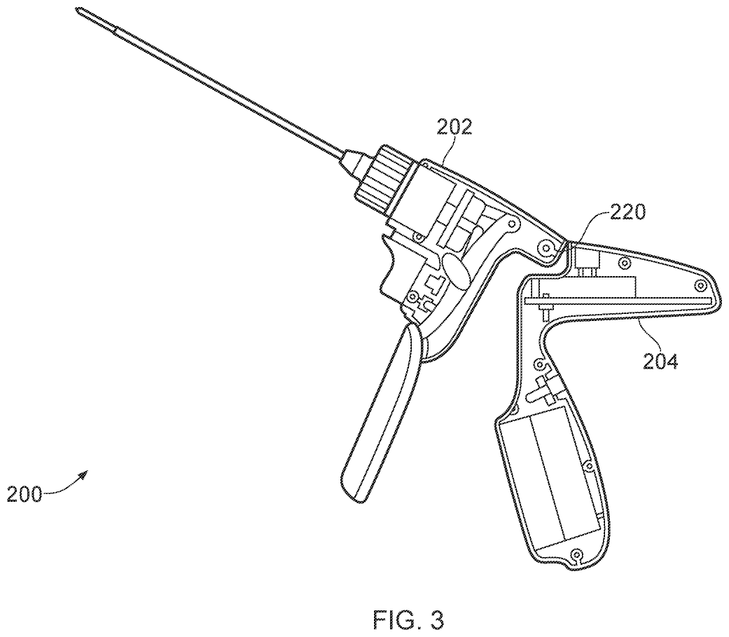

FIG. 3 illustrates the electrosurgical instrument 200 in the process of being connected. The end effector unit 202 and the electronics unit 204 may be connected initially from the top, where there may be a clip 220.

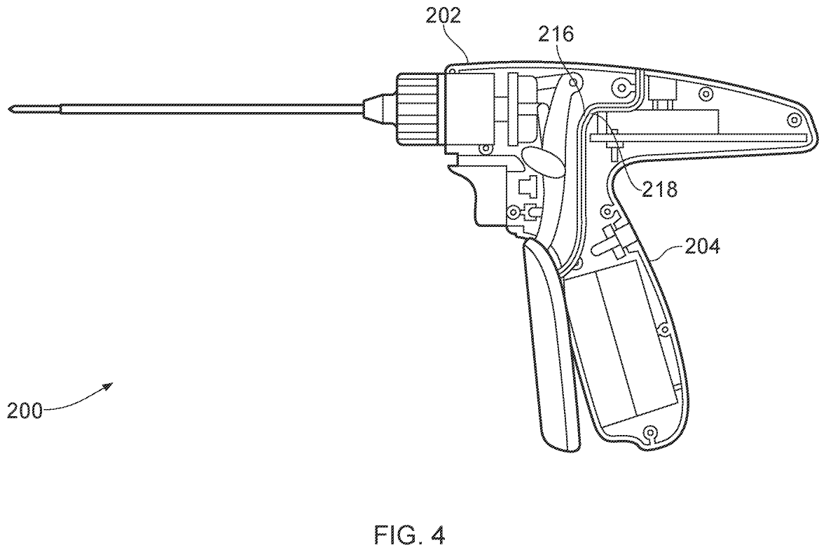

FIG. 4 illustrates the electrosurgical instrument 200 fully connected and ready to be used by a clinician during surgery. The end effector unit 202 is connected to the electronics unit 204 along the complementary interfaces 216, 218 of the units. The interfaces are user-releasable, such that the end effector unit 202 and the electronics unit 204 may be connected and disconnected by a user. The interface 216 of the end effector unit 202 sits flush with the interface 218 of the electronics unit.

Various modifications whether by way of addition, deletion, or substitution of features may be made to the above described embodiment to provide further embodiments, any and all of which are intended to be encompassed by the appended claims.

1. A reusable electronics unit for use in an electrosurgical instrument, the electrosurgical instrument in use comprising an end effector unit including an end effector, and the reusable electronics unit, the reusable electronics unit comprising:
a radio frequency (RF) electrosurgical signal generator circuitry for supplying in use RF electrosurgical signals to the end effector;
wherein the reusable electronics unit is sealed, the reusable electronics unit further comprising a user-releasable electrical interface connectable in use to a complementary electrical interface provided on the end effector unit through which the RF electrosurgical signals are supplied in use.

2. The reusable electronics unit according to claim 1, wherein the reusable electronics unit is hermetically sealed.

3. The reusable electronics unit according to claim 1, wherein the reusable electronics unit further comprises a battery.

4. The reusable electronics unit according to claim 3, wherein the electronics unit further comprises inductive charging circuitry, such that the battery of the reusable electronics unit may be charged inductively.

5. The reusable electronics unit according to claim 1, wherein there is no mechanical drive between the reusable electronics unit and the end effector unit.

6. An end effector unit for use in an electrosurgical instrument, the electrosurgical instrument in use comprising the end effector unit and a sealed reusable electronics unit, the reusable electronics unit including a radio frequency (RF) electrosurgical signal generator circuitry for supplying in use RF electrosurgical signals to the end effector, the end effector unit comprising:
an end effector;
wherein the end effector unit further comprises a user-releasable electrical interface connectable in use to a complementary electrical interface provided on the reusable electronics unit through which the RF electrosurgical signals are supplied in use.

7. The end effector unit according to claim 6, wherein the end effector unit further comprises a movable mechanism capable of receiving user input, such that the operation of the electrosurgical end effector is altered by movement of the mechanism

8. The end effector unit according to claim 6, wherein the end effector unit is a single-use unit.

9. The end effector unit according to claim 6, wherein there is no mechanical drive between the reusable electronics unit and the end effector unit.

10. An electrosurgical instrument, comprising:
the reusable electronics unit according to claim 1; and
an end effector unit for use in an electrosurgical instrument, the electrosurgical instrument in use comprising the end effector unit and a sealed reusable electronics unit, the reusable electronics unit including a radio frequency (RF) electrosurgical signal generator circuitry for supplying in use RF electrosurgical signals to the end effector, the end effector unit comprising:
an end effector;
wherein the end effector unit further comprises a user-releasable electrical interface connectable in use to a complementary electrical interface provided on the reusable electronics unit through which the RF electrosurgical signals are supplied in use;
wherein the interface of the reusable electronics unit is connected to the interface of the end effector unit.

11. The electrosurgical instrument according to claim 10, wherein all moving parts of the electrosurgical instrument are contained within the end effector unit.

12. An electrosurgical instrument comprising a first part and a second part, the first part being permitted a limited number of uses and the second part being re-usable,
the first part comprising an end effector including one or more electrosurgical electrodes, and associated mechanical actuation componentry including user-operable componentry to control the end effector via the associated mechanical actuation componentry, the arrangement being such that the first part is not sealed such that it is capable of being contaminated internally by surgical debris when the end effector is in use;
the second part comprising RF electrosurgical signal generation circuitry for supplying in use RF electrosurgical signals to the electrosurgical electrodes of the end effector, the second part being sealed to prevent the ingress of surgical debris when in use to prevent internal contamination by surgical debris.

13. An electrosurgical instrument according to claim 12, wherein the first part is single-use.

14. An electrosurgical instrument according to claim 12, wherein there is no mechanical drive between the first part and the second part.

15. An electrosurgical instrument according to claim 12, wherein the second part is hermetically sealed.
